(19) United States
(12) Patent Application Publication
(10) Pub. No.: US 2007/0104782 A1
(43) Pub. Date: May 10, 2007

(54) Modified release tablet formulations with enhanced mechanical properties

(76) Inventors: Melissa E. Ibrahim, Owings Mills, MD (US); Amir H. Shojaei, Phoenixville, PA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/494,759

(22) Filed: Jul. 28, 2006

Related U.S. Application Data
(60) Provisional application No. 60/703,000, filed on Jul. 28, 2005.
(60) Provisional application No. 60/702,982, filed on Jul. 28, 2005.

(51) Int. Cl. A61K 31/4745; A61K 31/138; A61K 9/20; A61K 9/22

(52) U.S. Cl. 424/464; 424/468; 514/283; 514/651

(57) ABSTRACT

A method of formulating a drug in solid dosage form of a specified hardness, the drug containing at least one pharmaceutically active agent that has a pH dependent release profile, at least one non-pH dependent sustained release agent, and an effective amount of Eudragit L100-55.

### RELATED APPLICATIONS

This application claims the benefit of the filing date of U.S. Provisional Application Ser. No. 60/703,000 filed Jul. 28, 2005, which is incorporated by reference herein.

This application is also related to previously filed provisional application 60/702,982 filed Jul. 28, 2005. The entire contents of the foregoing patent application is expressly incorporated by reference in its entirety.

### FIELD OF THE INVENTION

The present invention relates to a formulation that provides a robust (in terms of compressibility) pharmaceutical matrix tablet. The robustness is obtained through the use of excipients not previously known to enhance mechanical properties of a tablet. The formulation is suited for delivery of a pharmaceutically active agent or agents in a sustained (modified and sustained are used interchangeably in this text) release manner.

### BACKGROUND OF THE INVENTION

Matrix tablets usually contain excipients that are functionally necessary for certain characteristics of the tablet, for instance compressibility, flowability, friability, and modified release. In some cases, excipients will work in a unique way by unexpectedly affecting a tablet characteristic.

This phenomenon has been recognized in the field. For example, U.S. Pat. No. 6,358,525 states that some excipients used as sustained release polymers, such as hydroxypropylcellulose and methylcellulose, can improve the hardness of tablets as well as provide sustained release. Other patents note sugars as compressible fillers (U.S. Pat. No. 6,221,392), and silicone dioxide and methylcellulose as compressible excipients (U.S. Pat. No. 6,358,533).

U.S. Pat. No. 6,287,599 and U.S. Pat. No. 6,811,794, related to guanfacine, incorporated herein by reference, disclose sustained release pharmaceutical compositions with a minimized pH dependent or a pH-independent dissolution profile. The compositions are useful for pharmaceutically active agents that are pH dependent in order to obtain a good sustained release profile. Although formulations containing a certain methacrylic acid copolymer are shown (Eudragit L® 100-55), there is no indication that this particular polymer can be used to obtain robust tablets capable of achieving sufficient or preferred hardness of a matrix tablet, and it is not so used.

The chemical name of guanfacine hydrochloride is N-amidino-2-(2,6-dichlorophenyl) acetamide hydrochloride and its has the molecular formula, C₉H₁₀Cl₃N₃O.

### SUMMARY OF THE INVENTION

The present invention in one aspect relates to a method of formulating a drug in a solid dosage form of a specified hardness which comprises at least one pharmaceutically active agent that has a pH dependent release profile and at least one non-pH dependent sustained release agent, the method comprising selecting an amount of Eudragit L100-55 specifically to achieve said specified hardness and incorporating said amount of Eudragit L100-55 into the drug.

In another aspect the invention relates to a method of adjusting the hardness of a pharmaceutical formulation which comprises at least one pharmaceutically active agent that has a pH dependent release profile and at least one non-pH dependent sustained release agent, the method comprising (a) adding a first amount of Eudragit L100-55 to said pharmaceutical formulation, forming a tablet and then testing the hardness of the tablet to obtain a first hardness value, (b) adding at least one second amount of Eudragit L100-55 to said pharmaceutical formulation, forming at least one second tablet and then testing the hardness of the at least one second tablet to obtain at least one second hardness value, followed by (c) selecting an amount of Eudragit L100-55 for said pharmaceutical formulation which achieves a desired hardness for tablets made from the formulation.

The present invention further relates to a pharmaceutical composition comprising a pharmaceutically active agent and a specific methacrylic acid copolymer. The methacrylic acid copolymer is useful not only for the required modified release property of the product, but also for sufficient compressibility.

Tablet formulations have to meet certain performance criteria, which are often conflicting, such as high strength or hardness but acceptable dissolution. As it pertains to this invention, the target minimum average hardness of the tablet formulation should be at least, for example, 4.5 kP, e.g., 6 kP, and below 9.5, which can be a desired hardness, for the appropriate dissolution profile. But depending on the drug involved, other values are also achievable by the invention. These parameters are necessary for a viable product that is expected to pass friability testing as well as attain a desired sustained release dissolution profile while maintaining processibility and scalability.

Modified release polymers are necessary for a formulation according to the present invention, in order to confer the appropriate in vivo release profile. Methacrylic acid copolymer, more specifically Eudragit L100-55 (Rohm America, Inc.) is used to target release in the duodenum portion of the small intestine (i.e., about pH 5.5). It has now been found that the presence of this excipient in the pharmaceutical formulations of the present invention produces a tablet within the necessary range of hardness.

The present invention also relates to a method of treating behavioral disorders with sustained release tablet formulations described herein containing guanfacine. According to U.S. Pat. No. 5,854,290, incorporated herein by reference, guanfacine is useful for the treatment of Attention Deficit Hyperactivity Disorder, Conduct Disorder, Oppositional Defiant Disorder, Tourette's Syndrome, Lesch-Nyan Syndrome, and the disinhibitory symptoms associated with Post-Traumatic Stress Syndrome and dementia. The sustained release formulations of the present invention will allow for once-a-day dosing to increase convenience and subject compliance, significantly reduce peak-to-trough fluctuations that will improve subject tolerability, and provide effective extended duration of effect.

Finally, the present invention includes a process for preparing the tablets described herein.

### BRIEF DESCRIPTION OF THE DRAWINGS

FIG. 1 shows the range of hardness at different compression forces for formulations with different Eudragit polymers.

FIG. 2 shows the results of hardness tests at various compression forces of formulations containing either Eudragit L100-55 or Eudragit RSPO.

### DETAILED DESCRIPTION OF THE INVENTION

The invention relates to sustained release pharmaceutical compositions. More particularly, this invention relates to the presence of a certain methacrylic acid copolymer in sustained release pharmaceutical compositions that have a pH-independent or a minimized pH-dependent dissolution profile. Particularly, the compositions of the present invention include the methacrylic acid copolymer, Eudragit L100-55. Eudragit L® 100-55 is (poly(methacrylic acid, ethyl acrylate)), (anionic polymer of methacrylic acid and methacrylates—Methacrylic copolymer Type C, NF) marketed by Rohm America, Inc. All specification sheets available from Rohm America, Inc. by the filing date of this application for Eudragit L® 100-55 are hereby incorporated herein by reference.

The invention relates to a method of formulating a drug in a solid dosage form of a specified hardness which comprises at least one pharmaceutically active agent that has a pH dependent release profile and at least one non-pH dependent sustained release agent, the method comprising selecting an amount of Eudragit L100-55 specifically designed to achieve said specified hardness and incorporating said amount of Eudragit L100-55 into the drug.

In particular, such a modified release pharmaceutical tablet composition includes at least one pharmaceutically active agent that has a pH dependent release profile, at least one non-pH dependent sustained release agent, and Eudragit L100-55, which is present at a range of about 25 to about 45 wt. %, preferably from about 25 percent to about 40 percent, most preferably from about 26, 27, 28, etc. percent to about 32, 33, 34, 35, etc. percent (w/w) of the total composition. Lower or higher amounts will also be useful in conjunction with various drugs. The active agent(s) has (have) a solubility profile wherein the active agent(s) is (are) more soluble in an acidic medium than in a basic medium.

The rate at which a drug goes into solution when it is dissolved in a medium is proportional to the solubility of the drug in the medium. Many drugs have different solubilities at different pHs. These pH-dependent solubility differences lead to pH-dependent dissolution profiles. In general, pH-dependent dissolution is an undesirable product characteristic.

Compressed matrix tablets containing a basic drug often give a faster dissolution profile in simulated gastric fluid, having a pH of about 1.0, than in simulated intestinal fluid (pH 6.8 to 7.4).

It is an object of the present invention to provide a pharmaceutical composition with a minimized pH dependent or a pH-independent dissolution profile, and which displays sufficient hardness to make an acceptable tablet product.

The tablet composition of the present invention comprises at least one pharmaceutically active agent that has a pH dependent release profile, at least one non-pH dependent sustained release agent, and about 25 to about 40 wt % of Eudragit L100-55. The lower percentage is the minimal amount needed to attain sufficient hardness while maintaining an appropriate dissolution profile. Preferably, Eudragit L100-55 is present in the tablet composition at about 33%, or one-third of the formulation.

Although the concept of the present invention is found to be particularly useful in a formulation of guanfacine hydrochloride, it is contemplated that the invention is applicable to any other formulations of pharmaceutically active agents that would benefit from the release characteristics of the polymer as well as the hardness conferred thereby. For example, these include pharmaceutically active agents that are pH dependent and which may be included in the composition, including, but are not limited to, weakly basic drugs and their salts that have higher solubilities at lower pH levels. Such drugs include, for example, guanfacine hydrochloride, guanadrel sulfate, riserpine, anagrelide hydrochloride, propanolol, metoprolol, atenolol, timolol, erthyrthromycin, clonidine, chlorpheniramine, bromopheniramine, diltiazen, and scopolamine. In general, the pharmaceutically active agent is present in the composition in an amount of from about 0.1 wt. % to about 70 wt. %, preferably from about 1 wt. % to about 40 wt. %.

In one embodiment, the at least one pharmaceutically active agent is guanfacine hydrochloride. Preferably, guanfacine free base is present in the composition in an amount of about 0.1% to about 5 wt. %, preferably 0.25-5% (w/w), more preferably 0.3-4% (w/w), 0.33-3.5% (w/w), 0.5-3% (w/w), 0.75-2% (w/w), etc. More preferably, guanfacine hydrochloride tablets are in dosages of about 1.14 mg. to about 4.6 mg. guanfacine HCl per tablet, or about 1 to about 4 mg. free base per tablet. Most preferably, the tablets of guanfacine are in doses of 1, 2, 3 and 4 mg. free base.

Non-pH-dependent sustained release agents which may be included in the composition include, but are not limited to, ethylcellulose, cellulose acetate, vinyl acetate/vinyl chloride copolymers, (non-pH dependent) acrylate/methacrylate copolymers, polyethylene oxide, hydroxypropyl methylcellulose, carrageenan, alginic acid and salts thereof, hydroxyethyl cellulose, hydroxypropyl cellulose, karaya gum, acacia gum, tragacanth gum, locust bean gum, guar gum, sodium carboxymethyl cellulose, methyl cellulose, beeswax, carnauba wax, cetyl alcohol, hydrogenated vegetable oils, and stearyl alcohol. In general, the at least one non-pH-dependent sustained release agent is present in the composition in an amount of from about 5 wt. % to about 50 wt. %, preferably from about 10 wt. % to about 30 wt. %. It is to be understood, however, that the scope of the present invention is not to be limited to any particular non-pH-dependent sustained release agents.

Agents that increase the solubility of the at least one pharmaceutically active agent at a pH greater than 5.5 are optionally present in the compositions of the present invention. Such agents include, but are not limited to, organic acids. Such organic acids maintain an acidic microenvironment in the tablet, and include, but are not limited to, citric acid, fumaric acid, tartaric acid, adipic acid, glucono delta-lactone, and malic acid.

The composition of the present invention may further include other materials such as bulking agents, disintegrating agents, anti-adherants and glidants, lubricants, and binding agents.

Bulking agents include, but are not limited to, microcrystalline cellulose (eg., Avicel®, FMC Corp., Emcocel®, Mendell Inc., Prosolv HD90, Penwest Corp.), mannitol, xylitol, dicalcium phosphates (eg. Emcompress, Mendell Inc.) calcium sulfate (eg. Compactrol, Mendell Inc.) starches, lactose, sucrose (Dipac, Amstar, and Nutab, Ingredient Technology), dextrose (Emdex, Mendell, Inc.), sorbitol, cellulose powder (Elcema, Degussa, and Solka Floc, Mendell, Inc.) The bulking agent may be present in the composition in an amount of from about 5 wt. % to about 90 wt. %, preferably from about 10 wt. % to about 50 wt. %.

Disintegrating agents that may be included in the composition include, but are not limited to, microcrystalline cellulose, starches, crospovidone (eg. Polyplasdone XL, International Specialty Products.), sodium starch glycolate (Explotab, Mendell Inc.), and crosscarmellose sodium (e.g., Ac-Di-Sol, FMC Corp.). The disintegrating agent may be present in the composition in an amount of from about 0.5 wt. % to about 30 wt. %, preferably from about 5 wt. % to about 20 wt. %.

Antiadherants and glidants may be employed in the compositions of the present invention. These include, but are not limited to, talc, corn starch, silicon dioxide, sodium lauryl sulfate, and metallic stearates. The antiadherant or glidant may be present in the composition in an amount of from about 0.2 wt. % to about 10 wt. %, preferably from about 0.5 wt. % to about 3 wt. %.

Lubricants that may be present in the composition include, but are not limited to, magnesium stearate, calcium stearate, sodium stearate, stearic acid, sodium stearyl fumarate, hydrogenated cotton seed oil (sterotex), talc, and waxes, including but not limited to, beeswax, carnuba wax, cetyl alcohol, glyceryl stearate, glyceryl palmitate, glyceryl behenate, hydrogenated vegetable oils, and stearyl alcohol. The lubricant may be present in an amount of from about 0.2 wt. % to about 20 wt. %, preferably from about 5 wt. % to about 15 wt. %.

Binding agents which may be employed include, but are not limited to, polyvinyl pyrrollidone, starch, methylcellulose, hydroxypropyl methylcellulose, carboxymethyl cellulose, sucrose solution, dextrose solution, acacia, tragacanth and locust bean gum. The binding agent may be present in the composition in an amount of from about 0.2 wt. % to about 20 wt. %, preferably from about 5 wt. % to about 15 wt. %.

The compositions of the present invention are preferably made by a direct compression method, or by a wet granulation method. Preferred is dry blending followed by direct compression or wet granulation followed by drying and direct compression, especially for moisture-sensitive active agents such as guanfacine. In the direct compression method, the at least one pharmaceutically active agent and other ingredients are sieved through a stainless steel screen, such as a 40 mesh screen. The sieved materials then are charged to a suitable blender, and blended for about 10 minutes with an intensifier bar on for about 3 minutes. The blend then is compressed into tablets on a rotary press using appropriate tooling. The compressed tablets may be coated, if desired.

In the wet granulation method, the at least one pharmaceutically active agent and other ingredients are granulated with a granulating fluid (e.g., isopropyl alcohol, ethyl alcohol, and water) in a planetary mixer, high shear mixer, or fluidized bed granulator. Binding agents may be contained in the granulating fluid, or may be in the dry mix. The wet granules are dried in an oven or fluidized bed dryer, and then sieved through a suitable screen to obtain free flowing granules. The resulting granules were blended with a suitable lubricant and glidant, and the lubricated granules are compressed into tablets on a rotary press using appropriate tooling. If desired, a coating can be applied onto the compressed tablets.

Preferably, the formulations are dry-blended and direct-compressed. Tablets of acceptable hardness (minimum 6 kP) are produced only when Eudragit L100-55 was included in the formulations. Several variations were tested, as shown below. Some known hardness enhancing excipients, i.e., titanium dioxide and silicone dioxide, were used to try to produce a viable formulation without Eudragit L100-55 present, and it was not possible.

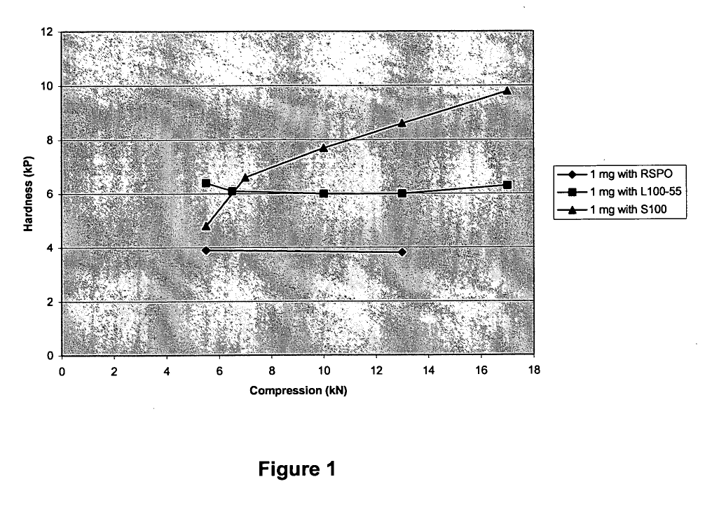

Identical formulations were used for investigations, with the only difference being that one would contain Eudragit L100-55, Eudragit RSPO, or Eudragit S100. Ammonio methacrylate copolymer, Eudragit RSPO, another excipient used in the formulations to provide controlled release, did not produce a compressible formulation of appropriate hardness. Eudragit RSPO is a pH independent polymer that has a low permeability for controlled release formulations. In general, the formulations containing Eudragit RSPO had poor flow and compressibility. Hardness enhancers did not improve the hardness of formulations containing Eudragit RSPO, but they also proportionately increased the hardness of the formulations with Eudragit L100-55. Eudragit S100 is a pH dependent anionic polymer solubilizing above pH 7.0 for targeted drug delivery in the ileum. When this polymer was incorporated into the formulation, hardness of the tablet was very sensitive to changes in the processing compression. Small changes in compression caused a large change in hardness; this leads to problems during processing, especially during scale-up and production. For instance, at a common compression setting range of 6-7 kN, the hardness of the tablet could vary from 4.5 to 7 kP. This process would be difficult to set controllable parameters. Acceptable compression ranging is to have minimal tablet hardness changes with main compression force adjustments. See further, FIG. 1.

When the pharmaceutically active agent is guanfacine hydrochloride, the composition may be employed in treating a behavioral disorder, such as attention deficit disorder, or attention deficit with hyperactivity disorder. The composition including guanfacine hydrochloride is administered to an animal, such as a mammal, including human and non-human primates, in an amount effective to treat the disorders mentioned hereinabove. Preferably, the amount effective for treating the behavior disorder is from about 1 to about 4 mg. guanfacine free base per day.

The compositions of the present invention may be employed to treat a variety of diseases or disorders.

When guanfacine hydrochloride is administered as part of a composition in accordance with the present invention, there is a reduction in the number of side effects associated with the administration of guanfacine hydrochloride, or a reduction in the likelihood of side effects associated with the administration of guanfacine hydrochloride.

The invention now will be described with respect to the following examples; however, the scope of the present invention is not intended to be limited thereby.

### EXAMPLES

### Example 1

The primary pieces of equipment used for manufacturing the tablets are a 16 quart V-shaped blender equipped with an intensifier bar and a 16 station rotary tablet press. All materials are passed through a 40 mesh screen and charged into a 16 quart V-blender, with guanfacine sandwiched in the middle. The mix is blended for ten minutes, with the intensifier bar turned on for minutes 5-8. The blend is charged into a polyethylene bag and then transferred to the hopper of the Stokes tablet press. The blend is compressed to the appropriate hardness for the necessary tablet weight. Tablet hardness is tested with a Schleuniger hardness tester.

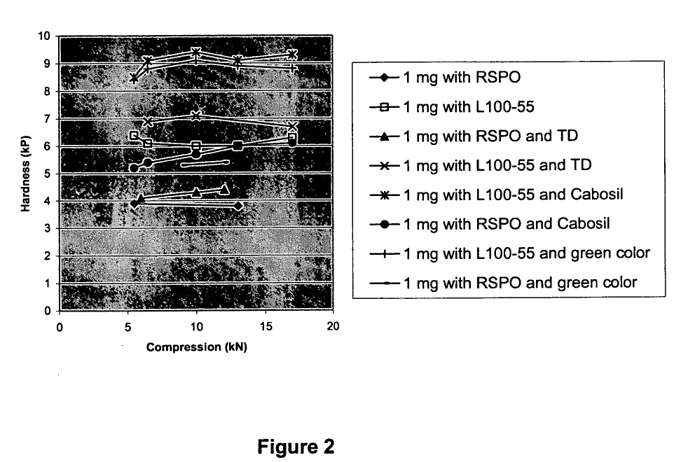

Formulations containing either Eudragit L100-55 or Eudragit RSPO were tested for hardness at various compression forces (see FIG. 2).

TABLE 1

| | | Composition |
|---|---|---|
| Ingredients | Composition 1 (% w/w) | (% w/w) 2 |
| Guanfacine HCl | 0.76 | 0.76 |
| Methocel K4M* | 13.34 | 13.34 |
| Eudragit L100-55* | 33.33 | N/A |
| Eudragit RSPO | N/A | 33.33 |
| Fumaric acid | 5.00 | 5.00 |
| Compritol 888 ATO* | 13.33 | 13.33 |
| Ludipress* | 16.91 | 16.91 |
| Prosolv HD90* | 17.33 | 17.33 |

*Methocel K4M - Hydroxypropylmethylcellulose, Dow Chemical;
Eudragit L100-55 - poly(methacrylic acid, ethyl acrylate), a methacrylic acid copolymer, Rohm America, Inc.;
Eudragit RSPO - Ammonio Methacrylate copolymer, Rohm America, Inc.;
Compritol 888 ATO - Glyceryl dibehenate, Gattefosse;
Ludipress - Lactose/povidone/crospovidone, BASF;
Prosolv HD90 - Silicified microcrystalline cellulose, Penwest.

TABLE 2

| Composition | Average Hardness (kP) |
|---|---|
| 1 (Eudragit L100-55) | 6.2 |
| 2 (Eudragit RSPO) | 3.9 |

### Example 2

Formulations were tested by adding colorant to examine its effect on hardness (see FIG. 2). To the final blends of the formulations of Table 1 was added 0.5% Green Pigment. Results of hardness testing are shown in Table 3.

TABLE 3

| Composition | Average Hardness (kP) |
|---|---|
| 1 (Eudragit L100-55) | 8.8 |
| 2 (Eudragit RSPO) | 5.4 |

### Example 3

Formulations were tested by adding titanium dioxide, a component of some colorants (see FIG. 2).

TABLE 4

| Ingredients | Composition (% w/w) 3 | Composition (% w/w) 4 |
|---|---|---|
| Guanfacine HCl | 0.76 | 0.76 |
| Methocel K4M | 13.34 | 13.34 |
| Eudragit L100-55 | 33.33 | N/A |
| Eudragit RSPO | N/A | 33.33 |
| Fumaric acid | 5.00 | 5.00 |
| Compritol 888 ATO | 13.33 | 13.33 |
| Ludipress | 16.41 | 16.41 |
| Prosolv HD90 | 17.33 | 17.33 |
| Titanium dioxide | 0.50 | 0.50 |

TABLE 5

| Composition | Average Hardness (kP) |
|---|---|
| 3 (Eudragit L100-55) | 6.9 |
| 4 (Eudragit RSPO) | 4.3 |

### Example 4

Silicone dioxide, a hardness enhancer, was added to the formulations (see FIG. 2).

TABLE 6

| Ingredients | Composition (% w/w) 5 | Composition (% w/w) 6 |
|---|---|---|
| Guanfacine HCl | 0.76 | 0.76 |
| Methocel K4M | 13.34 | 13.34 |
| Eudragit L100-55 | 33.33 | N/A |
| Eudragit RSPO | N/A | 33.33 |
| Fumaric acid | 5.00 | 5.00 |
| Compritol 888 ATO | 13.33 | 13.33 |
| Ludipress | 16.41 | 16.41 |
| Prosolv HD90 | 17.33 | 17.33 |
| Silicone dioxide | 0.50 | 0.50 |

TABLE 7

| Composition | Average Hardness (kP) |
|---|---|
| 5 (Eudragit L100-55) | 9.1 |
| 6 (Eudragit RSPO) | 5.7 |

It is to be understood that the scope of the present invention is not to be limited to the specific embodiments described above. The invention may be practiced other than as particularly described and still be within the scope of the accompanying claims.

What is claimed is:

1. A method of formulating a drug in a solid dosage form of a specified hardness which comprises at least one pharmaceutically active agent that has a pH dependent release profile and at least one non-pH dependent sustained release agent, the method comprising selecting an amount of Eudragit L100-55 specifically designed to achieve said specified hardness and incorporating said amount of Eudragit L100-55 into the drug.

2. A method of adjusting the hardness of a pharmaceutical formulation which comprises at least one pharmaceutically active agent that has a pH dependent release profile and at least one non-pH dependent sustained release agent, the method comprising (a) adding a first amount of Eudragit L100-55 to said pharmaceutical formulation, forming a tablet and then testing the hardness of the tablet to obtain a first hardness value, (b) adding at least one second amount of Eudragit L100-55 to said pharmaceutical formulation, forming at least one second tablet and then testing the hardness of the at least one second tablet to obtain at least one second hardness value, followed by (c) selecting an amount of Eudragit L100-55 for said pharmaceutical formulation which achieves a desired hardness for tablets made from the formulation.

3. A method according to claim 1, wherein the amount of Eudragit L100-55 is about 26 to 40% by weight of the total dosage form.

4. A method according to claim 1, wherein said at least one pharmaceutically active agent is guanfacine hydrochloride, guanfacine, anagrelide, guanethidine monosulfate, guanadrel sulfate, riserpine, propanolol, metoprolol, atenolol, timolol, erythromycin, clonidine, chlorpheniramine, bromopheniramine, diltiazem, or scopolamine.

5. A method according to claim 1, wherein said non-pH dependent sustained release agent is selected from the group consisting of ethylcellulose, cellulose acetate, vinyl acetate/vinyl chloride copolymers, acrylate/methacrylate copolymers, polyethylene oxide, hydroxypropyl methylcellulose, carageenan, alginic acid and salts thereof, hydroxyethyl cellulose, hydroxypropyl cellulose, karaya gum, acacia gum, tragacanth gum, locust bean gum, guar gum, sodium carboxymethyl cellulose, methyl cellulose, beeswax, carnauba wax, cetyl alcohol, hydrogenated vegetable oils, and stearyl alcohol.

6. A method according to claim 1, wherein the drug further comprises a binding agent.

7. A method according to claim 6, wherein said binding agent is selected from the group consisting of polyvinyl pyrrolidone, starch, methylcellulose, hydroxypropyl methylcellulose, carboxymethyl cellulose, sucrose solution, dextrose solution, acacia, tragacanth, and locust bean gum.

8. A method according to claim 1, wherein said pharmaceutically active agent is present in the composition in an amount of from about 0.1 wt. % to about 5 wt. %.

9. A method according to claim 1, wherein said non-pH-dependent sustained release agent is present in the composition in an amount of from about 5 wt. % to about 50 wt. %.

10. A method according to claim 9, wherein said non-pH-dependent sustained release agent is present in the composition in an amount of from about 10 wt. % to about 30 wt. %.

11. A method according to claim 1, wherein said Eudragit L100-55 is present in the composition in an amount of about 33 wt. %.

12. A method according to claim 1, wherein said non-pH-dependent sustained release agent is ethylcellulose or hydroxypropyl methylcellulose.

13. A method according to claim 1, wherein the drug further comprises a bulking agent.

14. A method according to claim 13, wherein said bulking agent is microcrystalline cellulose.

15. A method according to claim 1, wherein the drug further comprises a lubricant.

16. A method according to claim 15, wherein said lubricant is glyceryl dibehenate, magnesium stearate, or sodium stearyl fumarate.

17. The method according to claim 1, wherein the tablet has an average hardness of at least 6 kP.

18. The method according to claim 1, wherein the tablet has an average hardness of 6 kP to 9.5 kP.
